(19) United States
(12) Patent Application Publication
(10) Pub. No.: US 2007/0136921 A1
(43) Pub. Date: Jun. 21, 2007

(54) Attachment for an item of protective sports gear

(76) Inventor: Lonnie Dale Tiegs, Johannesburg (ZA)

Correspondence Address: FITCH EVEN TABIN AND FLANNERY, 120 SOUTH LA SALLE STREET, SUITE 1600, CHICAGO, IL, 60603-3406, US

(21) Appl. No.: 10/582,566

(22) PCT Filed: Dec. 10, 2004
(86) PCT No.: PCT/IB04/04064

§ 371 Date: Jun. 12, 2006

(30) Foreign Application Priority Data
Dec. 12, 2003 (ZA) 2003/9689
May 6, 2004 (ZA) 2004/3445

(51) Int. Cl. A61F 9/00

(52) U.S. Cl. 2/10

(57) ABSTRACT

Air-deflection attachment for an item of protective gear worn on the head of a wearer. The attachment includes a deflector portion of unitary substantially air-impermeable flexible construction for location alongside the face and forwardly from an ear of the wearer, and an attachment portion attached to the deflector portion for releasably attaching the attachment to the item of protective gear. The deflector portion is generally in the form of a vertically elongated aerofoil, for deflecting flow of air past the head of the wearer arising from movement of the wearer in a direction in which the wearer is facing, away from the wearer's outer ear canal opening.

This invention relates to protective sports gear. It more particularly relates to an air-deflection attachment for an item of protective sports gear. It relates also to a set of air-deflection attachments for an item of protective sports gear, and to a protective sports gear assembly.

It is expected that the invention will particularly advantageously be applicable to outdoor sports such as cycling, in-line skating or rollerblading, snowboarding, skiing, yachting, and the like. Accordingly, such application should particularly be borne in mind when considering this specification.

In accordance with one aspect of the invention there is provided an air-deflection attachment for an item of protective sports gear worn on the head of a wearer, the attachment including:

a deflector portion of unitary substantially air-impermeable flexible construction for location alongside the face and forwardly from an ear of a wearer of the item of protective gear, the deflector portion being generally in the form of a vertically elongated aerofoil, having an operatively inner face and an operatively outer face, the inner face and the outer face meeting along an operatively leading edge of the deflector portion and respectively extending between said leading edge and an operatively trailing edge of the deflector portion, the inner face providing an inner edge spaced laterally inwardly from the trailing edge for substantially sealingly abutting a side of the face of the wearer, and the outer face being for deflecting flow of air past the head of a wearer arising from movement of the wearer in a direction in which the wearer is facing, the deflection being away from the wearer's outer ear canal opening, and acting to reduce wind noise levels experienced by the wearer when moving; and
an attachment portion attached to the deflector portion for releasably attaching the attachment to the item of protective gear.

It will be appreciated that in sports such as, for example, in-line skating or rollerblading, snowboarding, skiing, and yachting, where a sportsperson's head does not always necessarily face in the direction in which the sportsperson moves, each attachment will also act to deflect flow of air past the head of the sportsperson away from the sportsperson's outer ear canal opening, when the sportsperson's head is, within limits, turned sideways.

The deflector portion may be in the form of a moulding of resiliently flexible plastics foam construction, the flexible construction of the deflector portion permitting conformation of the inner edge thereof to the shape of the side of the face of a wearer of the item of protective gear.

The inner edge of the deflector portion may be provided by a ridge extending longitudinally along at least part of the inner face of the deflector portion, the ridge being spaced from the leading edge of the deflector portion and spaced from the trailing edge of the deflector portion, part of the deflector portion forming a forward extension of the deflector portion from the ridge, and the attachment portion being attached to said extension.

Naturally, the vertical length and the shape of the attachment may be customized by being selected in accordance with the length and shape of a particular sportsperson's ears, and in accordance with an expected or actual angle of attack, which angle of attack varies from sport to sport, which the sportsperson's head is expected to make with air flow past the sportsperson's head. The size and the shape of the attachment may further be customized in accordance with personal auditory preference of sportspeople, an in accordance with the particular construction of an item of protective sports gear to which the attachment is intended to be attached.

The attachment portion may be of unitary construction, being releasably attached to the forward extension of the deflector portion.

In one embodiment, the attachment may be for attachment to a protective sports helmet, the attachment portion including at least one clip for attaching the attachment to a strap of the sports helmet, each clip including a pair of limbs fast with each other at the leading edge of the deflector portion and projecting rearwardly from the leading edge, one limb being located on the operatively inner face of the forward extension of the deflector portion and the other limb being located on the operatively outer face of the extension, with the extension being sandwiched between the limbs of each clip, the extension being provided with at least one opening spaced from the leading edge of the deflector portion and spaced from said ridge, through which aperture at least one of the limbs of each clip extends, the free ends of the limbs of each clip being releasably securable together, to permit the strap of the helmet to be received between the extension and one of the limbs of each clip.

Each attachment portion may include a plurality of said clips longitudinally spaced in series along the length of the extension, the attachment portion also including a spine formation connecting the plurality of clips together, the extension being provided with a corresponding plurality of longitudinally spaced openings through which one limb of each clip extends.

The attachment portion may also be in the form of a moulding of flexible synthetic plastics material.

In another embodiment, the attachment may be for attachment to a pair of glasses, the attachment portion including a clip for attaching the attachment to an ear shaft assembly of the pair of glasses, typically in use dark- or sunglasses, and a variation thereof can be constructed to be attachable to a pair of goggles.

In this embodiment, the attachment portion may include an elongated spine formation fast with and extending longitudinally along at least part of the vertical length of the extension, the clip comprising two limbs fast with the spine formation for receiving therebetween part of a said ear shaft assembly.

The spine formation may be of composite construction, having an inner component located on the inner face of the extension and from which inner component the clip projects, and an outer component located on the outer face of the extension, one said component being provided with a prong projecting laterally therefrom through an opening in the extension spaced from the leading edge of the deflector portion and spaced from said ridge, the other said component being provided with an aperture for securingly receiving the prong. Said one component may include a plurality of said prongs longitudinally spaced in series, the extension being provided with a corresponding plurality of said openings for respectively receiving the respective prongs, the other component being provided with a corresponding plurality of apertures for respectively securingly receiving the respective prongs, one of the components being provided with an operatively upwardly projecting restriction or stop formation for abutting the ear shaft assembly of the pair of glasses, to restrict pivoting of the lower end of the attachment away form the side of the face of a wearer of the pair of glasses about an axis extending in the fore and aft direction, parallel to the ear shaft of the glasses.

The attachment formation may be of resiliently flexible construction, the flexibility of the spine formation increasing towards the operatively lower end thereof, to promote conformation of the inner edge of the deflector portion with a side of the face of the wearer of a pair of glasses to which the attachment is attached. The inner component and the outer component may be in the form of mouldings of flexible synthetic plastics material.

In accordance with another embodiment of the invention there is provided a set of attachments for an item of protective gear, the set including two attachments as hereinbefore described, one of the attachments being shaped for association with the left ear of a wearer of the item of protective gear, the other attachment being shaped for association with the right ear of the wearer.

In accordance with a further aspect of the invention there is provided a protective sports gear assembly which comprises:
an item of protective sports gear worn on the head of a wearer; and
a pair of air-deflection attachments attached to opposites sides of the item of protective sports gear, each air-deflection attachment being an attachment as hereinbefore described, one attachment being shaped for association with left ear of a wearer of the item of protective sports gear, and the other attachment being shaped for association with the right ear of the wearer.

In one embodiment, the item of protective sports gear may be a protective sports helmet having chin straps, each air-deflection attachment being an attachment as hereinbefore described for attachment to a protective sports gear helmet, each attachment being attached to a chin strap of the helmet.

In another embodiment, the item of sports gear may be a pair of glasses, each air-deflection attachment being an attachment as hereinbefore described for attachment to a pair of glasses, each attachment being attached to an ear shaft assembly of the pair of glasses.

The glasses may be dark- or sunglasses.

The invention is now described, by way of example, with reference to the accompanying diagrammatic drawings.

In the drawings:

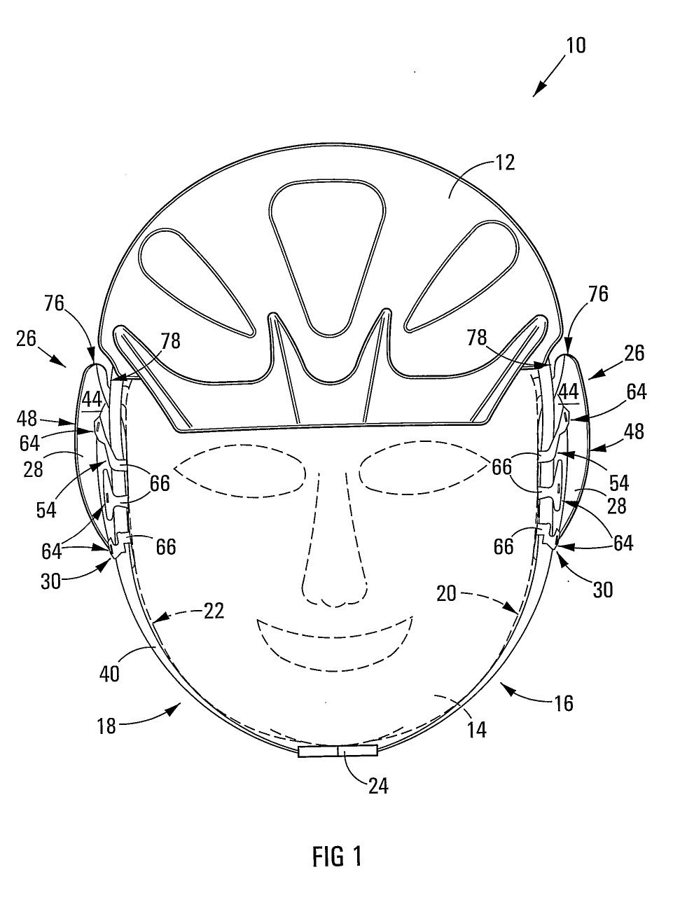

FIG. 1 is a schematic front elevation of one embodiment of a sports gear assembly in accordance with the invention;

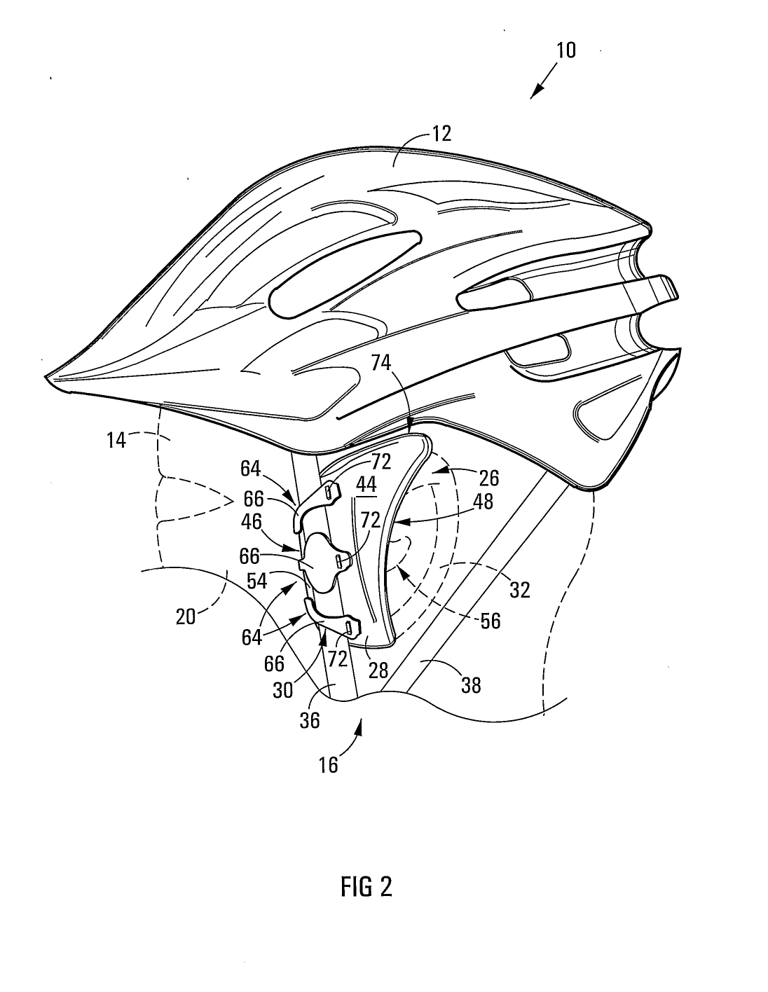

FIG. 2 is a schematic side elevation of the sports gear assembly shown in FIG. 1;

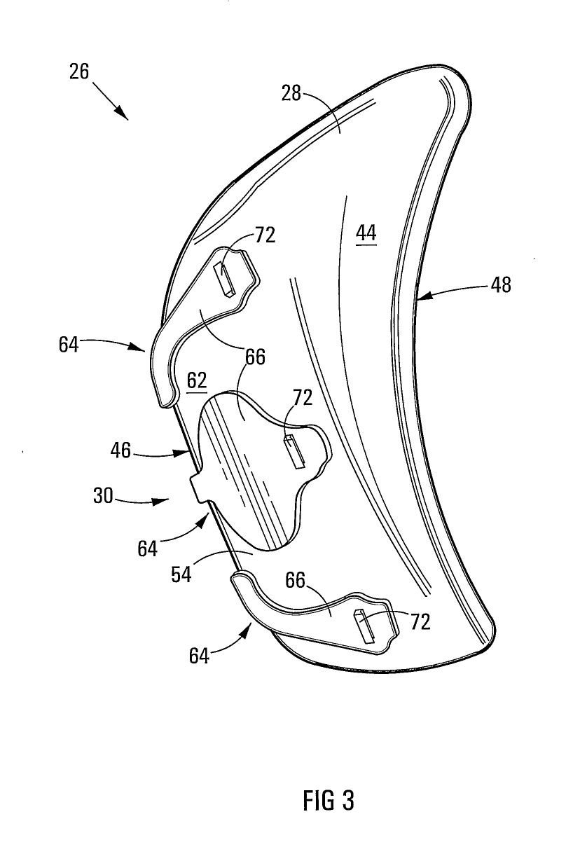

FIG. 3 is a schematic face-on elevation, showing an outer face, of an air-deflection attachment in accordance with the invention, the air-deflection attachment forming part of the assembly shown in FIGS. 1 and 2;

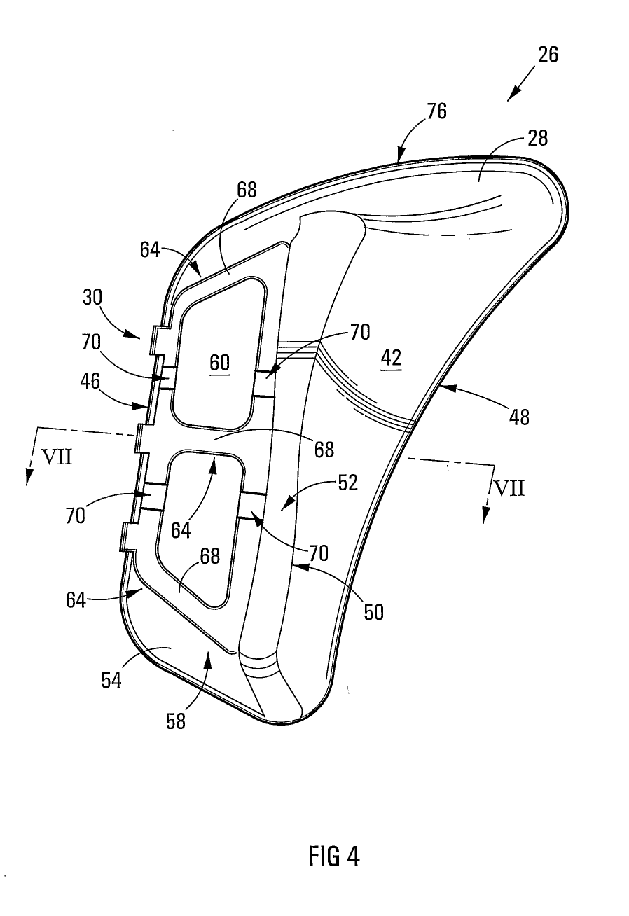

FIG. 4 is a schematic face-on elevation, showing an inner face, of the air-deflection attachment of FIG. 3;

FIG. 5 is a schematic top plan view of the attachment of FIG. 3;

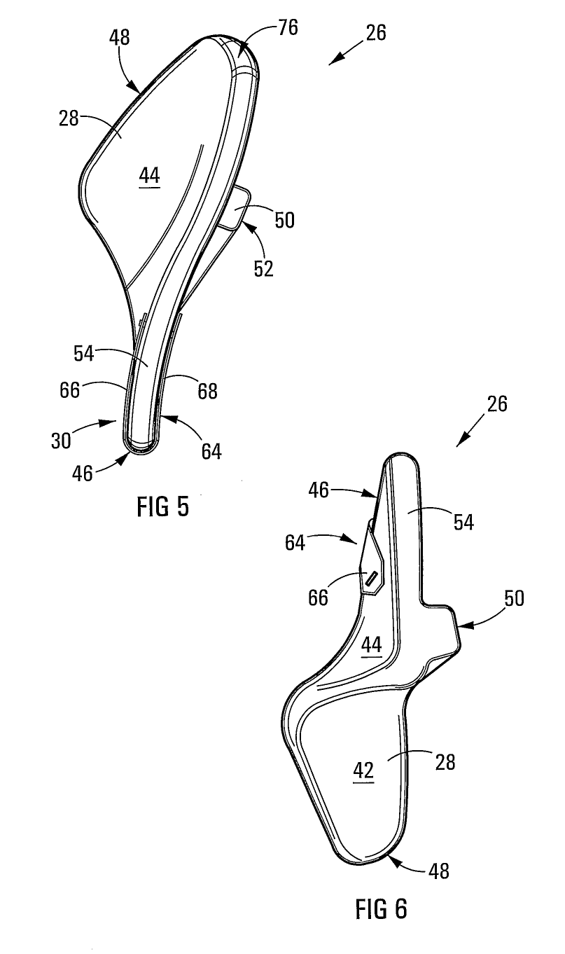

FIG. 6 is a schematic bottom plan view of the attachment of FIG. 3;

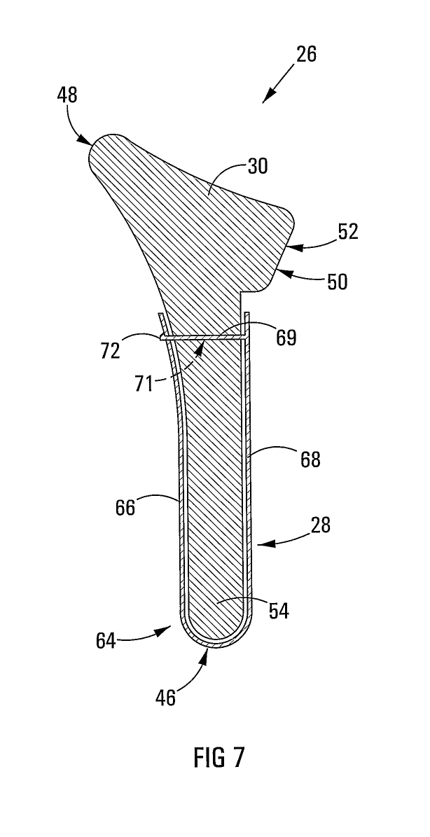

FIG. 7 is a schematic sectional top plan view of the assembly shown in FIG. 5, the section being taken at VII-VII in FIG. 4;

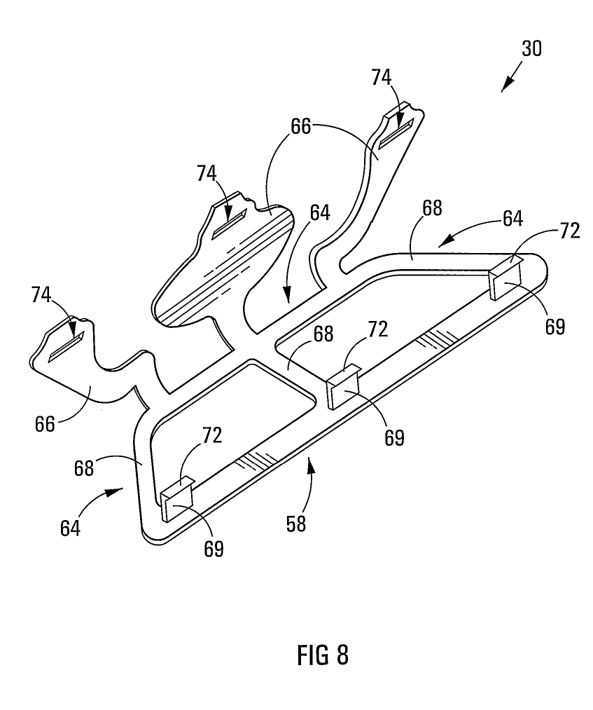

FIG. 8 is a three-dimensional view of an attachment portion forming part of the air-deflection attachment of FIG. 3;

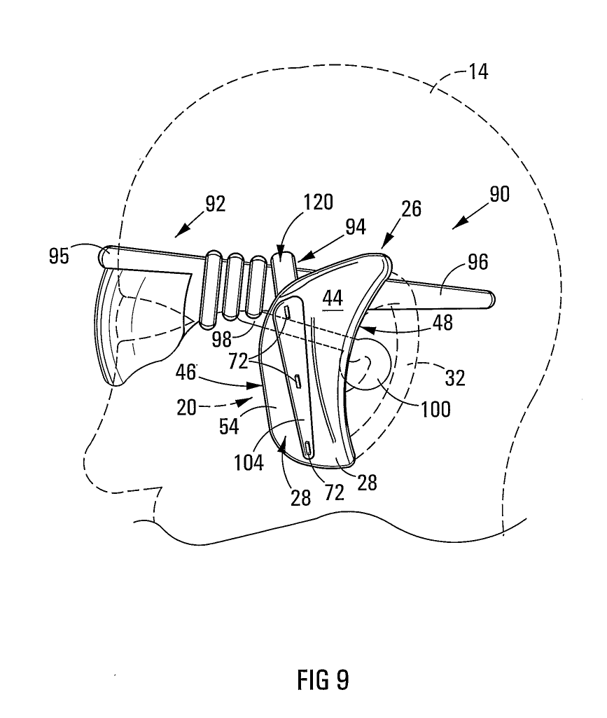

FIG. 9 is a schematic side elevation of another embodiment of a sports gear assembly in accordance with the invention;

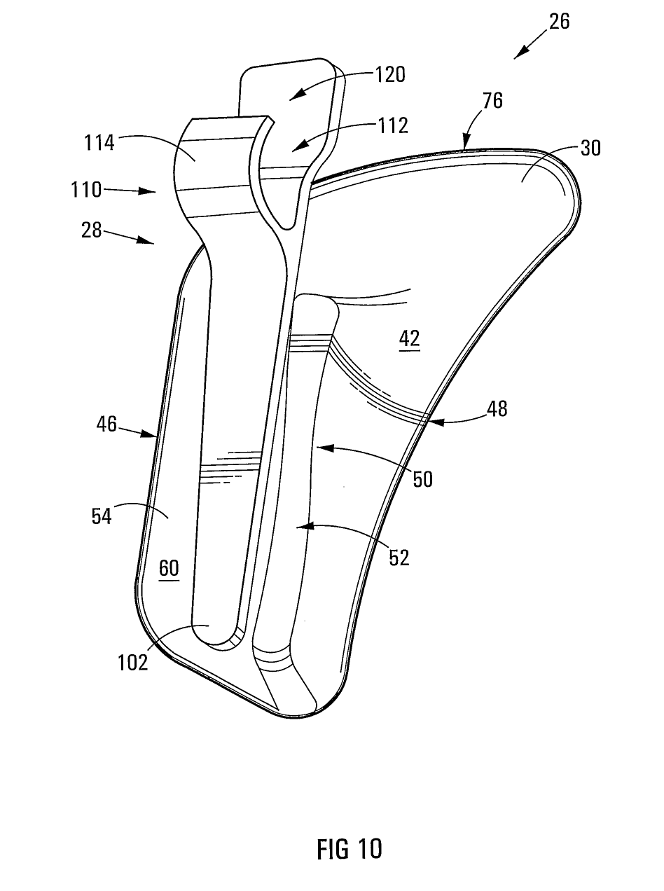

FIG. 10 is a schematic face-on elevation, showing an inner face, of another embodiment of an air-deflection attachment in accordance with the invention, the air-deflection attachment forming part of the assembly shown in FIG. 9; and

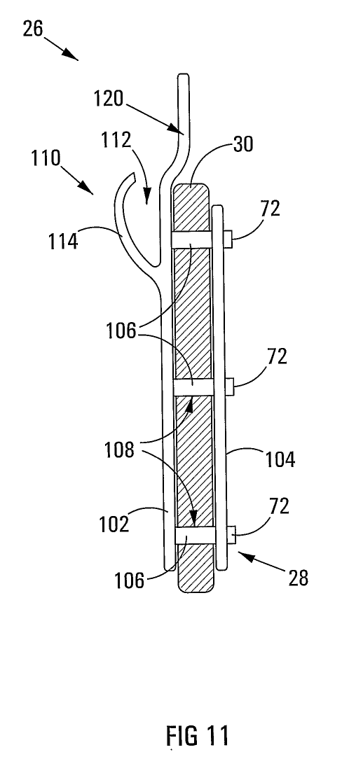

FIG. 11 is a front elevation of the attachment shown in FIG. 10, with a deflector portion forming part of the attachment being shown in section and in concept only.

With reference to FIGS. 1 and 2 of the drawings, a sports gear assembly in accordance with the invention is generally indicated by reference numeral 10. The assembly 10 includes a cycling helmet 12 received on the head 14 (shown schematically in broken lines) of a sportsperson, in this case a cyclist, for protecting the head of the cyclist. The cycling helmet 12 is secured to the head 14 of the cyclist by means of chin strap assemblies 16, 18, respectively extending alongside the respective sides 20, 22 of the face of the cyclist, the strap assemblies 16, 18 having free ends meeting under the chin of the cyclist and being secured together by means of a clip 24.

The assembly 10 further includes two air-deflection attachments (see in particular FIGS. 3-7) in accordance with the invention, each of the air-deflection attachments, for ease of reference, being indicated by reference numeral 26. The attachments 26 are respectively releasably attached to the respective strap assemblies 16, 18. Each of the attachments 26 comprises a deflector portion 28 for deflecting air, as hereinafter described, and an attachment portion 30 (FIG. 8) releasably attached to the deflector portion 28 and by means of which each attachment 26 is releasably attached to the associated strap assembly 16, 18, as hereinafter described.

The attachment portions 28 are mirror images of each other in shape and size and are attached to the strap assemblies 16, 18, such that they are respectively located somewhat forwardly of the respective ears, only one of which is shown in broken lines FIG. 2 and is designated by reference numeral 32, of the cyclist. As will be appreciated, the attachments 26 may be constructed such that the deflector portions 28 are, in some embodiments, spaced further forwardly than shown in the drawings of the ears 32 of the cyclist. Although the ears 32 of the cyclist are hidden behind the attachments 26 in FIG. 1, the attachments 26 need not necessarily, in order to be effective, be sized to hide the entire ears 32 of the cyclist, when seen from the front, as will become more apparent hereinafter. The attachments 26 can thus, in some embodiments be smaller, even substantially smaller, than shown in the drawings.

The strap assemblies 16, 18 each comprise a pair of downwardly converging chin straps, the straps of the strap assembly 16 being indicated by reference numerals 36 and 38, and the one strap of the strap assembly 18 which is visible, being indicated by reference numeral 40. The straps of each strap assembly 16, 18 are adjustable in length, respectively being spaced in the fore- and aft direction from the respective ears 32 of the cyclist. Thus, as can be seen in FIG. 2, when seen in side view, each pair of said straps forms a downwardly pointing V-shape, with the ear 32 of the cyclist being located between the straps 36, 38 and with the other ear of the cyclist being located between the strap 40 and the other strap of the strap assembly 18, which strap is not visible. The attachments 26 are respectively attached to the straps 36, 40, i.e. the straps which are located in front of the ears 32 of the cyclist.

Referring back to the attachments 26, the deflector portions 28 are shaped and sized such that they deflect flow of air past the head 14 of the cyclist arising from movement of the cyclist in a direction in which the cyclist faces, the deflection being away from the cyclist's ears 32, thereby reducing wind noise levels or volumes experienced by the cyclist while cycling. Naturally, when the cyclist is cycling in cold weather, the attachments 26 also serve to shelter the cyclist's ears 26, 28 from the cold air moving past the cyclist's head 14.

The deflector portions 28 are of unitary more or less air-impermeable flexible plastics foam construction. In this embodiment, the deflector portions 28 are in the form of mouldings or castings manufactured from viscoelastic plastics foam. The foam plastics from which the deflector portions 28 are constructed can, if desired, include an ultra-violet protecting agent and an antioxidant for resisting damage to the deflector portions 28 caused by exposure of the attachments 26 to sunlight and to damp environments.

The deflector portions 28 are generally in the form of vertically elongated smoothly-contoured aerofoils, each deflector portion 28 having an inner face 42 and an outer face 44. The inner face 42 and the outer face 44 of each deflector portion 28 meet along a leading edge 46 of the deflector portion 28 and extend between the leading edge 46 and a trailing edge 48 of the deflector portion 28. The inner face 42 of each deflector portion 28 is shaped to define a ridge-like formation 50 providing an inner edge 52 of the deflector portion 28. The ridge-like formation 50 and, accordingly, the inner edge 52 of each deflector portion 28 in use extends more or less vertically, between the leading edge 46 and the trailing edge 48, along part of the inner face 42 of the deflector portion 28. The inner edges 52 of the deflector portions 28, in use, as can be seen in FIG. 1, more or less sealingly abut the respective sides 20, 22 of the face of the cyclist. Thus, in use, the outer face 44 deflects the air moving past the head 14 of the cyclist away form the cyclist's ears 32. Further, the trailing edges 48 of the deflector portions 28 are respectively spaced laterally outwardly from the inner edges 52 and, accordingly, also from the respective sides 20, 22 of the face of the cyclist. By virtue of the resiliently flexible construction of the deflector portions 28, their inner edges 52 more or less conform with the profiles of the sides 20, 22 of the face of the cyclist, to effect said more or less sealing abutment.

With reference also to FIGS. 3-7, parts of the deflector portions 28 forwardly of the ridge-like formations 50 define forward extensions 54 of the deflector portions 28. The forward extensions 54 are spaced laterally outwardly from and project forwardly from the inner edges 52, with the attachment portions 30 being releasably secured to the forward extensions 54. Operatively front edges of the forward extensions 54 thus define the leading edges 46 of the deflector portions 28.

As can be seen in FIG. 2, the width of the deflector portions 28 is such that, when seen in side elevation, their trailing edges 48 are forward of and shy of the outer ear canal openings 56 (shown in broken lines) of the cyclist, so that, when seen in side elevation, the outer ear canal openings 56 of the cyclist are exposed. Also, when seen in side elevation, the width of the deflector portions 28 is such that at a position about one third of their length downward from their upper edges (described hereinafter), the deflector portions 28 cover at most a quarter of the width of the ears 32 of the cyclist. In most applications, however, the deflector portions 28 will be located and shaped such that a greater part, than that shown in the drawings, of each ear 32 of the cyclist is exposed in use, when seen from the side. Typically, bigger deflection portions 28 will be located further forwardly of the ears 32 of a cyclist than smaller deflector portions 28.

Each deflector portion 28 is, as mentioned, vertically elongated and is long enough to ensure deflection of flow of air past the head 14 of the cyclist, resulting from movement in a direction in which the cyclist faces, away from more or less the entire associated ear 32 of the cyclist.

Naturally, the vertical length and the shape of the deflector portions 28 can be customized by being selected in accordance with the length and shape of a particular sportsperson's ears, and in accordance with an expected or actual angle of attack which the head 14 of a sportsperson is expected to make with air flow past the sportperson's head 14. It will be appreciated that said angle of attack varies from sport to sport, so that the deflector portions 28 may be differently shaped and sized for different sports.

Each attachment portion 30 includes an elongated flexible synthetic plastics spine formation 58 which, in this embodiment, extends lengthwise partially along the inner face 60 of its associated extension 54. Naturally, the spine formations 58 can, in other embodiments (not shown) extend along the outer faces 62, or even along the front edges of the extensions 54. Furthermore, each attachment portion 30 includes three clips 64 fast with and longitudinally spaced in series from one another along the length of the spine formation 58. Each clip 64 includes a pair of opposed limbs 66, 68, the limbs 66 being located on the outer face 62 of the associated extension 54, and the limbs 68, which in this embodiment are defined by parts of the spine formation 58, are located on the inner face 60 of the associated extension 54, such that the extension 54 is sandwiched between the limbs 66, 68 of each clip 64. Ends of the limbs 68, defined by prongs 69 (FIGS. 7 and 8) projecting laterally from the spine formation 58, extend through openings 71 (FIG. 7) through the extensions 54, said prongs 69 being provided with clip formations 72 which are releasably received in apertures 74 (FIG. 8) provided in the free ends of the limbs 66.

The spine formation 58 is provided with relatively thinner sections 70 (shown only in FIG. 4 and omitted from FIG. 8), which sections 70 are provided between the limbs 68, to enhance the flexibility of the attachment portion 30 along its length.

The straps 36, 40 are snugly received between the outer faces 62 of the extensions 54 and the limbs 66 of the clips 64, so that the clips 64 are snugly longitudinally slidable along their associated straps 36, 40, to facilitate adjustment of the vertical positions of the attachments 26 relative to the ears 32 of the cyclist.

The attachment portions 30, like the deflector portions 28, are of unitary plastics construction and, because they are releasably attached to the deflector portions 28, the deflector portions 28 and the attachment portions 30 are independently replaceable when worn or broken.

The rib-like formations 50 terminate shy of upper edges 76 of the deflector portions 28, so that, when the deflector portions 28 are seen in front elevation, stepped recesses or rebates 78 (FIG. 1) are defined adjacent their upper edges 66. In use, these recesses or rebates 78 provide for receipt of ear shafts of spectacles or sunglasses or the straps of goggles (not shown).

Referring now to FIG. 9 of the drawings, a further embodiment of a sports gear assembly in accordance with the invention is generally indicated by reference numeral 90. The sports gear assembly 90 in certain aspects resembles the sports gear assembly 10 and, accordingly, unless otherwise indicated, parts or features of the assembly 90 similar to or resembling parts or features of the assembly 10, are indicated by the same reference numerals used for the assembly 10.

The protective sports gear assembly 90 includes an item of protective sports gear which in this embodiment is in the form of a pair of sports sunglasses 92 for protecting the eyes of the wearer of the pair of sunglasses 92. Naturally, the pair of glasses need not necessarily be sunglasses, but could be any other type of glasses or goggles worn on the head of a sportsperson. The pair of sunglasses 92 has ear shaft assemblies, only one of which is visible and is indicated by reference numeral 94, secured to and projecting operatively rearwardly from a frame 95 of the pair of sunglasses 92. The attachments 26 are respectively attached to the respective ear shaft assemblies 94 of the pair of sunglasses 92, as hereinafter described.

As can be seen in FIG. 9, the attachments 26 are located in more or less similar positions relative to the ears 32 of the wearer of the assembly 90 as the attachments 26 of the protective sports gear assembly 10 and, accordingly, serve the same purpose when the wearer of the assembly 90 moves in a direction in which the wearer faces.

More particularly, in this embodiment, each ear shaft assembly 94 of the pair of sunglasses 92 includes an elongated ear shaft 96 which has embedded therein a digital music player, and an earphone shaft 98 projecting rearwardly and downwardly from a forward part of the ear shaft 96, the earphone shaft 98 carrying, at a free rear end thereof, an earphone 100. The earphones 100 comprise loudspeakers for receipt in the outer ear canal openings 56 of the wearer of the assembly 90.

In this embodiment, the attachment portions 30 are of composite construction, each attachment portion 30 including an elongated inner component 102 located on the inner face 60 of the forward extension 54 of the deflector portion, and an outer component 104 located on the outer face 62 of the forward extension 54, the components 102, 104 together providing an elongated spine formation. The inner component 102 is provided with three laterally projecting longitudinally spaced prongs 106, the prongs 106 projecting through openings 108 (FIG. 11) provided in the extension 54. The openings 108 are spaced from the leading edge 46 and from the ridge-like formation 50 of the deflector portion 28. The outer component 104, on the other hand, is provided with three longitudinally spaced apertures (not visible) in which free ends 72 (see also FIG. 8) of the prongs 106 are releasable received, the deflector portion 28 (shown in concept for illustrative purposes only in FIG. 11) being sandwiched between the inner component 102 and the outer component 104, thereby securing the attachment portion 30 to the deflector portion 28.

Furthermore, an elongated upwardly extending curved limb 114 projects from an operatively inner face of the inner component 102. The base of the limb 114 is spaced operatively downwardly from an upper end of the inner component 102, and the limb 114 is located more or less alongside the upper part of the inner component 102. The limb 114 and said upper part of the inner component 102 above the base of the limb (described further hereunder) comprise a clip 110 which, in turn, defines a receiving space 112, within which the earphone shaft 98 of one of the ear shaft assemblies 94 is snugly received, to attach the attachment 26 to the pair of sunglasses 92. To this end, the ridge-like formation 50 of the deflector portion 28 shown in FIGS. 9-11 is shorter than that of the deflector portion 28 shown in FIGS. 1-7, to provide space for the earphone shaft 98. In the embodiment shown, the clip 110 is upwardly opening, such that the attachment 26 is clipped on to the earphone shaft 98 form under the earphone shaft 98. As will be appreciated, in other embodiments (not shown), the clip 110 may be shaped such that the receiving space 112 opens downwardly, or even laterally away from the inner component 102, in which case the inner component 102 may include another limb projecting laterally therefrom. Naturally, the shape of the clip 110 will depend on the shape of the ear shaft assemblies of the pair of sunglasses to which the attachments 26 are to be attached.

As can be seen in FIGS. 9-11, the inner component 102 is somewhat longer than the outer component 104, such that the inner component 102 projects operatively upwardly from the upper edge 76 of the deflector portion 28, the upwardly projecting part thereof, which also forms part of the clip 110, forming a restriction formation or stop 120. The restriction formation or stop 120 abuts the ear shaft 96 of the pair of sunglasses 92, to restrict pivoting of the lower end of the attachment 26 about the earphone shaft 98 in a direction away from the associated side 20, 22 of the face of the wearer of the pair of sunglasses 92.

In practice, the attachments 26 shown in FIGS. 9-11 will be somewhat smaller than is shown in the drawings, typically, they will be sized to deflect air away from only the outer ear canal opening 56 of a wearer of the assembly 90, to reduce background noise to the music to which the wearer is listening, which background noise arises from movement of air past the wearer's head 14.

Furthermore, as will be appreciated, the clips 110 of the attachments 26 can, in embodiments where the pair of sunglasses 92 does not include the earphone shaft 98, be attached to the ear shafts 96 of the pair of sunglasses.

As can be seen in FIGS. 10 and 11, the inner component 102 and the outer component 104 are tapered in width towards their operatively lower ends. Thus, the width of the components 102,104 is reduced towards their lower ends, such that the stiffness thereof decreases towards their lower ends. This reduced stiffness promotes conformation of the inner edge 52 of each attachment 26 to the associated side 20, 22 of the face of the wearer of the sports gear assembly 90.

It will be appreciated that the attachments in accordance with the invention can also be used in combination with other forms of protective sports gear assemblies, such as snow goggles and the like, worn on the head- of a wearer, Likewise, the attachments can also be used with glasses or spectacles other than sunglasses.

The attachment in accordance with the invention is thus easily attached to and released form an item of protective sports gear, and does not, when attached to an item of sports gear, affect the normal intended functioning of such item.

Thus, the invention as described and illustrated, will, in practice, reduce wind noise levels or volumes experienced by a sportsperson during movement in a forward direction, thereby enabling the sportsperson more easily to identify other forms of noise such as traffic in the case of a cyclist. Although the Applicant does not wish to be bound by theory, it is believed that the particular construction of the attachments 26 will, in use, also serve to deflect, towards the sportsperson's ears, noise coming from the sportsperson's rear, thus enhancing the hearing, by the sportsperson, of noise from the rear.

1. An air-deflection attachment for an item of protective sports gear worn on the head of a wearer, the attachment including:
a deflector portion of unitary substantially air-impermeable flexible construction for location alongside the face and forwardly from an ear of a wearer of the item of protective gear, the deflector portion being generally in the form of a vertically elongated aerofoil, having an operatively inner face and an operatively outer face, the inner face and the outer face meeting along an operatively leading edge of the deflector portion and respectively extending between said leading edge and an operatively trailing edge of the deflector portion, the inner face providing an inner edge spaced laterally inwardly from the trailing edge for substantially sealingly abutting a side of the face of the wearer, the inner edge being provided by a ridge extending longitudinally along at least part of the inner face of the deflector portion, and the outer face being for deflecting flow of air past the head of a wearer arising from movement of the wearer in a direction in which the wearer is facing, the deflection being away from the wearer's outer ear canal opening, and acting to reduce wind noise levels experienced by the wearer when moving, the deflector portion being in the form of a moulding of resiliently flexible plastics foam construction, the flexible construction of the deflector portion permitting conformation of the inner edge thereof to the shape of the side of the face of a wearer of the item of protective gear; and
an attachment portion attached to the deflector portion for releasably attaching the attachment to the item of protective gear.

2. An attachment as claimed in claim 1, in which the ridge providing the inner edge of the deflector portion is spaced from the leading edge of the deflector portion and spaced from the trailing edge of the deflector portion, part of the deflector portion forming a forward extension of the deflector portion from the ridge, and the attachment portion being attached to said extension.

3. An attachment as claimed in claim 2, in which the attachment portion is of unitary construction, being releasably attached to the forward extension of the deflector portion.

4. An attachment as claimed in claim 3, which is for attachment to a protective sports helmet, the attachment portion including at least one clip for attaching the attachment to a strap of the sports helmet, each clip including a pair of limbs fast with each other at the leading edge of the deflector portion and projecting rearwardly from the leading edge, one limb being located on the operatively inner face of the forward extension of the deflector portion and the other limb being located on the operatively outer face of the extension, with the extension being sandwiched between the limbs of each clip, the extension being provided with at least one opening spaced from the leading edge of the deflector portion and spaced from said ridge, through which aperture at least one of the limbs of each clip extends, the free ends of the limbs of each clip being releasably securable together, to permit the strap of the helmet to be received between the extension and one of the limbs of each clip.

5. An attachment as claimed in claim 4, in which each attachment portion includes a plurality of said clips longitudinally spaced in series along the length of the extension, the attachment portion also including a spine formation connecting the plurality of clips together, the extension being provided with a corresponding plurality of longitudinally spaced openings through which one limb of each clip extends.

6. An attachment as claimed in claim 1, wherein the attachment portion is in the form of a moulding of flexible synthetic plastics material.

7. An attachment as claimed in claim 2, which is for attachment to a pair of glasses, the attachment portion including a clip for attaching the attachment to an ear shaft assembly of the pair of glasses.

8. An attachment as claimed in claim 7, in which the attachment portion includes an elongated spine formation fast with and extending longitudinally along at least part of the vertical length of the extension, the clip comprising two limbs fast with the spine formation for receiving therebetween part of a said ear shaft assembly.

9. An attachment as claimed in claim 8, in which the spine formation is of composite construction, having an inner component located on the inner face of the extension and from which inner component the clip projects, and an outer component located on the outer face of the extension, one said component being provided with a prong projecting laterally therefrom through an opening in the extension spaced from the leading edge of the deflector portion and spaced from said ridge, the other said component being provided with an aperture for securingly receiving the prong.

10. An attachment as claimed in claim 9, in which said one component includes a plurality of said prongs longitudinally spaced in series, the extension being provided with a corresponding plurality of said openings for respectively receiving the respective prongs, the other component being provided with a corresponding plurality of apertures for respectively securingly receiving the respective prongs, one of the components being provided with an operatively upwardly projecting restriction or stop formation for abutting the ear shaft assembly of the pair of glasses, to restrict pivoting of the lower end of the attachment away form the side of the face of a wearer of the pair of glasses about an axis extending in the fore and aft direction, parallel to the ear shaft of the glasses.

11. An attachment as claimed in claim 10, in which the attachment formation is of resiliently flexible construction, the flexibility of the spine formation increasing towards the operatively lower end thereof, to promote conformation of the inner edge of the deflector portion with a side of the face of the wearer of a pair of glasses to which the attachment is attached.

12. An attachment as claimed in claim 9, in which the inner component and the outer component are in the form of mouldings of flexible synthetic plastics material.

13. A set of attachments for an item of protective gear, the set including two attachments of claim 1, one of the attachments being shaped for association with the left ear of a wearer of the item of protective gear, the other attachment being shaped for association with the right ear of the wearer.

14. A protective sports gear assembly which comprises:
an item of protective sports gear worn on the head of a wearer; and
a pair of air-deflection attachments attached to opposites sides of the item of protective sports gear, each air-deflection attachment being an attachment as claimed in claim 1, one attachment being shaped for association with left ear of a wearer of the item of protective sports gear, and the other attachment being shaped for association with the right ear of the wearer.

15. A protective sports gear assembly as claimed in claim 14, wherein the item of protective sports gear is a protective sports helmet having chin straps, and each air-deflection attachment is attached to a chin strap of the helmet.

16. A protective sports gear assembly as claimed in claim 14, wherein the item of sports gear is a pair of glasses, each air-deflection attachment being attached to an ear shaft assembly of the pair of glasses.

17. A protective sports gear assembly as claimed in claim 15, wherein the attachment portion includes at least one clip for attaching the attachment to a strap of the sports helmet, each clip including a pair of limbs fast with each other at the leading edge of the deflector portion and projecting rearwardly from the leading edge, one limb being located on the operatively inner face of the forward extension of the deflector portion and the other limb being located on the operatively outer face of the extension, with the extension being sandwiched between the limbs of each clip, the extension being provided with at least one opening spaced from the leading edge of the deflector portion and spaced from said ridge, through which aperture at least one of the limbs of each clip extends, the free ends of the limbs of each clip being releasably securable together, to permit the strap of the helmet to be received between the extension and one of the limbs of each clip.

18. A protective sports gear assembly as claimed in claim 17, wherein each attachment portion includes a plurality of said clips longitudinally spaced in series along the length of the extension, the attachment portion also including a spine formation connecting the plurality of clips together, the extension being provided with a corresponding plurality of longitudinally spaced openings through which one limb of each clip extends.

19. A protective sports gear assembly as claimed in claim 16, wherein the attachment comprises a clip for attaching the attachment to an ear shaft assembly of a pair of glasses, and an elongated spine formation fast with and extending longitudinally along at least part of the vertical length of the extension, the clip comprising two limbs fast with the spine formation for receiving therebetween part of a said ear shaft assembly.

20. A protective sports gear assembly as claimed in claim 19, wherein the spine formation is of composite construction, having an inner component located on the inner face of the extension and from which inner component the clip projects, and an outer component located on the outer face of the extension, one said component being provided with a prong projecting laterally therefrom through an opening in the extension spaced from the leading edge of the deflector portion and spaced from said ridge, the other said component being provided with an aperture for securingly receiving the prong.
